(19) United States
(12) Patent Application Publication
(10) Pub. No.: US 2008/0033054 A1
(43) Pub. Date: Feb. 7, 2008

(54) Process for preparing memantine hydrochloride substantially free of impurities

(76) Inventors: Valeriano Merli, Cremella Lecco (IT); Paola Daverio, Milano (IT); Alessandra Vailati, Seregno(MI) (IT)

Correspondence Address: KENYON & KENYON LLP, ONE BROADWAY, NEW YORK, NY, 10004, US

(21) Appl. No.: 11/729,005

(22) Filed: Mar. 27, 2007

Related U.S. Application Data
(60) Provisional application No. 60/786,609, filed on Mar. 27, 2006.

(51) Int. Cl. A61K 31/13; A61P 25/16; A61P 25/28; C07C 211/38; G01N 30/02

(52) U.S. Cl. 514/662; 436/161; 564/459

(57) ABSTRACT

The present invention encompasses processes for preparing Memantine hydrochloride and its derivatives, substantially free of impurities.

### CROSS REFERENCE

This application claims the benefit of U.S. Provisional Patent Application No. 60/786,609, filed Mar. 27, 2006, which is incorporated herein by reference.

### FIELD OF THE INVENTION

The present invention encompasses processes for preparing Memantine hydrochloride and its derivatives, substantially free of impurities.

### BACKGROUND OF THE INVENTION

Memantine hydrochloride, 1-amino-3,5-dimethyladamantane hydrochloride, is one of a small group of drugs known as Tricyclic Antivirals (TAVs), and provides good and persistent activation of central nervous system N-methyl-d-aspartate (NMDA) receptors, such that it can be used in the treatment of Parkinson's and Alzheimer's diseases. The chemical structure of memantine hydrochloride is as illustrated below.

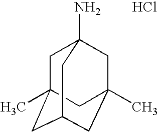

### Formula: C₁₂H₂₂CIN

### Molecular weight: 215.81

U.S. Pat. No. 3,391,142 (“'142 patent”) discloses the synthesis of memantine hydrochloride and its precursor, 1-acetamido-3,5-dimethyladamantane, according to following scheme. 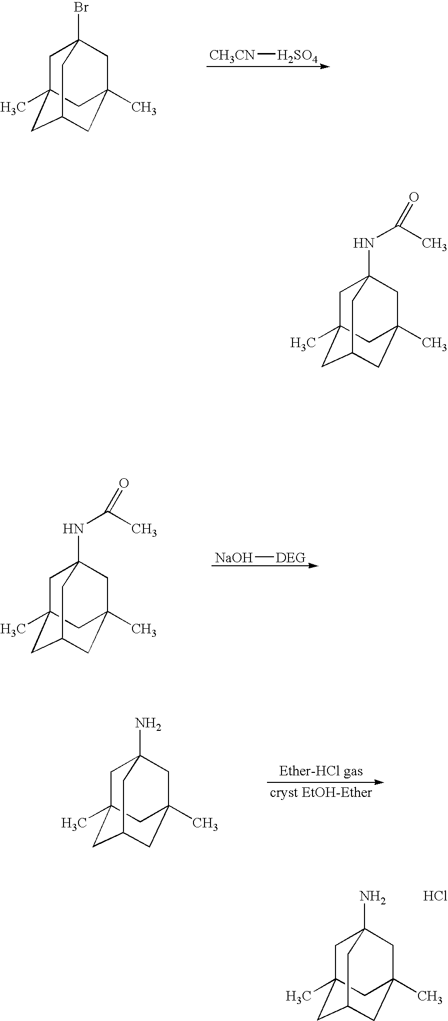
In the first reaction, 1-bromo-3,5-dimethyladamantane reacts with 17 moles of acetonitrile and 35 moles of sulphuric acid at room temperature to give the crude intermediate product in 100 percent yield. The intermediate product is subjected to alkaline hydrolysis with sodium hydroxide in diethylene glycol by refluxing at a temperature greater than 190° C. for six hours. The hydrolyzed product is diluted with water, followed by several benzene extractions, and the memantine free base is recovered by solvent distillation. The free base is then diluted with ether, and the addition of hydrogen chloride gas provides crude memantine hydrochloride. The crude product is then crystallized from a mixture of ethanol and ether.

The '142 patent also discloses the compounds: 1-bromo-3,5,7-trimethyladamantane (Br-TMAD) and 1-bromo-3-methyladamantane (Br-MMAD)

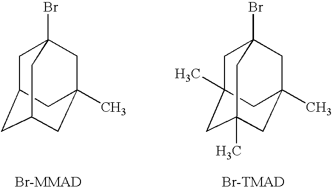

U.S. Pat. No. 5,061,703 also discloses the compounds: 1-Amino-3,5,7-trimethyladamantane hydrochloride (Me-MMN*HCl) and 1-Amino-3-methyladamantane hydrochloride (DesMe-MMN*HCl).

Like any synthetic compound, memantine hydrochloride salt can contain extraneous compounds or impurities that can come from many sources. They can be unreacted starting materials, by-products of the reaction, products of side reactions, or degradation products. Impurities in memantine hydrochloride salt or any active pharmaceutical ingredient (API) are undesirable and, in extreme cases, might even be harmful to a patient being treated with a dosage form containing the API.

It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic by-products, and degradation products.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the International Conference on Harmonization of Technical Requirements for Registration for Human Use (“ICH”) Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and by-products of the reaction and adjunct reagents used in the reaction will, in most cases, also be present in the product mixture. At certain stages during processing of the API, memantine hydrochloride, it must be analyzed for purity, typically, by HPLC, TLC or GC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and, thus, are as safe as possible for clinical use. As discussed above, in the United States, the Food and Drug Administration guidelines recommend that the amounts of some impurities be limited to less than 0.1 percent.

Generally, side products, by-products, and adjunct reagents (collectively “impurities”) are identified spectroscopically and/or with another physical method, and then associated with a peak position, such as that in a chromatogram, or a spot on a TLC plate. (Strobel p. 953, Strobel, H. A.; Heineman, W. R., Chemical Instrumentation: A Systematic Approach, 3rd dd. (Wiley & Sons: New York 1989)). Thereafter, the impurity can be identified, e.g., by its relative position in the chromatogram, where the position in a chromatogram is conventionally measured in minutes between injection of the sample on the column and elution of the particular component through the detector. The relative position in the chromatogram is known as the “retention time.”

The retention time can vary about a mean value based upon the condition of the instrumentation, as well as many other factors. To mitigate the effects such variations have upon accurate identification of an impurity, practitioners use the “relative retention time” (“RRT”) to identify impurities. (Strobel p. 922). The RRT of an impurity is its retention time divided by the retention time of a reference marker. It may be advantageous to select a compound other than the API that is added to, or present in, the mixture in an amount sufficiently large to be detectable and sufficiently low as not to saturate the column, and to use that compound as the reference marker for determination of the RRT.

Those skilled in the art of drug manufacturing research and development understand that a compound in a relatively pure state can be used as a “reference standard.” A reference standard is similar to a reference marker, which is used for qualitative analysis only, but is used to quantify the amount of the compound of the reference standard in an unknown mixture, as well. A reference standard is an “external standard,” when a solution of a known concentration of the reference standard and an unknown mixture are analyzed using the same technique. (Strobel p. 924, Snyder p. 549, Snyder, L. R.; Kirkland, J. J. Introduction to Modern Liquid Chromatography, 2nd ed. (John Wiley & Sons: New York 1979)). The amount of the compound in the mixture can be determined by comparing the magnitude of the detector response. See also U.S. Pat. No. 6,333,198, incorporated herein by reference.

The reference standard can also be used to quantify the amount of another compound in the mixture if a “response factor,” which compensates for differences in the sensitivity of the detector to the two compounds, has been predetermined. (Strobel p. 894). For this purpose, the reference standard is added directly to the mixture, and is known as an “internal standard.” (Strobel p. 925, Snyder p. 552).

The reference standard can serve as an internal standard when, without the deliberate addition of the reference standard, an unknown mixture contains a detectable amount of the reference standard compound using the technique known as “standard addition.”

In the “standard addition technique”, at least two samples are prepared by adding known and differing amounts of the internal standard. (Strobel pp. 391-393, Snyder pp. 571, 572). The proportion of the detector response due to the reference standard present in the mixture without the addition can be determined by plotting the detector response against the amount of the reference standard added to each of the samples, and extrapolating the plot to zero concentration of the reference standard. (See, e.g., Strobel, FIG. 11.4 p. 392). The response of a detector in GC or HPLC (e.g. UV detectors or refractive index detectors) can be and typically is different for each compound eluting from the GC or HPLC column. Response factors, as known, account for this difference in the response signal of the detector to different compounds eluting from the column.

As is known by those skilled in the art, the management of process impurities is greatly enhanced by understanding their chemical structures and synthetic pathways, and by identifying the parameters that influence the amount of impurities in the final product.

### SUMMARY OF THE INVENTION

In one embodiment the present invention provides a process for preparing memantine HCl having less than about 0.15% of one or both of Ac—NH-TMAD and Ac—NH-MMAD comprising measuring an amount of at least one or both of N-acetyl-1-amino-3,5,7-trimethyladamantane (Ac—NH-TMAD) and N-acetyl-1-amino-3-methyladamantane (Ac—NH-MMAD) in a batch of 1-acetamido-3,5-dimethyladamantane, selecting a batch of 1-acetamido-3,5-dimethyladamantane having less than about 0.15% of one or both of Ac—NH-TMAD or Ac—NH-MMAD and converting the selected batch of 1-acetamido-3,5-dimethyladamantane to memantine HCl containing less than about 0.15% of at least one of DesMe-MMN HCl or MeMMN HCl.

In one embodiment the present invention provides a process for preparing memantine HCl containing less than about 0.15% of at least one of DesMe-MMN HCl or MeMMN HCl comprising measuring an amount of one or both of 1-bromo-3,5,7-trimethyladamantane (Br-TMAD) or 1-bromo-3-methyladamantane (Br-MMAD) in a batch of 1-bromo-3,5-dimethyladamantane, selecting a batch having one or both of less than about 0.15% of Br-TMAD or less than about 0.20% area Br-MMAD and converting the batch of 1-bromo-3,5-dimethyladamantane to memantine HCl containing less than about 0.15% of at least one of DesMe-MMN HCl and MeMMN HCl.

In one embodiment the present invention provides a process for reducing amount of impurities present in memantine HCl comprising measuring an amount of at least one or both of 1-bromo-3,5,7-trimethyladamantane (Br-TMAD) and 1-bromo-3-methyladamantane (Br-MMAD) in a batch of 1-bromo-3,5-dimethyladamantane, selecting a batch having at least one of less than about 0.15% Br-TMAD or less than about 0.20% area Br-MMAD as measured by gas chromatography, and converting the batch of 1-bromo-3,5-dimethyladamantane to 1-acetamido-3,5-dimethyladamantane; measuring an amount of at least one of N-acetyl-1-amino-3,5,7-trimethyladamantane (Ac—NH-TMAD) and N-acetyl-1-amino-3-methyladamantane (Ac—NH-MMAD) in a batch of 1-acetamido-3,5-dimethyladamantane, selecting a batch of 1-acetamido-3,5-dimethyladamantane having less than about 0.15% area by gas chromatography of at least one of Ac—NH-TMAD and Ac—NH-MMAD and converting the selected batch of 1-acetamido-3,5-dimethyladamantane to memantine HCl containing less than about 0.15% of at least one of DesMe-MMN HCl and MeMMN HCl.

In one embodiment the present invention provides an isolated N-acetyl-1-amino-3,5,7-trimethyladamantane (Ac—NH-TMAD).

In one embodiment the present invention provides an isolated N-acetyl-1-amino-3-methyladamantane (Ac—NH-MMAD).

In one embodiment the present invention provides a method of determining the amount of an impurity in a sample of N-acetyl-1-amino-3,5-dimethyladamantane (Ac—NH-DMAD) comprising measuring by chromatography the area under a peak corresponding to at least one of N-acetyl-1-amino-3,5,7-trimethyladamantane (Ac—NH-TMAD) and N-acetyl-1-amino-3-methyladamantane (Ac—NH-MMAD) in a reference standard comprising a known amount of one or both of Ac—NH-TMAD and Ac—NH-MMAD; measuring by chromatography the area under a peak corresponding to Ac—NH-TMAD or Ac—NH-MMAD in a sample comprising Ac—NH-DMAD and at least one of Ac—NH-TMAD or Ac—NH-MMAD; and determining the amount of at least one of Ac—NH-TMAD and Ac—NH-MMAD in the sample by comparing the area of reference standard with that of the test sample.

In one embodiment the present invention provides a method of identifying an impurity in a sample of Ac—NH-DMAD comprising providing a reference marker sample of Ac—NH-TMAD or Ac—NH-MMAD, or two separate samples of each; determining by chromatography the relative retention time (RRT) corresponding to at least one of Ac—NH-TMAD or Ac—NH-MMAD in a sample comprising Ac—NH-DMAD, and Ac—NH-TMAD or Ac—NH-MMAD; and determining the relative retention time (RRT) of Ac—NH-TMAD or Ac—NH-MMAD in the sample by comparing the relative retention time (RRT) of the reference marker to the relative retention time (RRT) of the sample.

In one embodiment the present invention provides a process for preparing a pharmaceutical composition comprising memantine hydrochloride having less than about 0.15% of one or both Me-MMN*HCl or DesMe-MMN*HCl, comprising obtaining one or more batches of the compound memantine hydrochloride, measuring the level of either Me-MMN*HCl or DesMe-MMN*HCl in each of the samples, selecting a batch of memantine hydrochloride having less than about 0.15% of one or both of Me-MMN*HCl or DesMe-MMN*HCl, based on the measurement of the samples from the batches; and preparing from the selected batch a pharmaceutical composition comprising memantine hydrochloride and at least one pharmaceutically acceptable excipient.

### BRIEF DESCRIPTION OF THE DRAWINGS

FIG. 1 illustrates a Typical Chromatogram (GC) for Memantine Hydrochloride impurities.

FIG. 2 illustrates a Typical Chromatogram (GC) for 1-Acetamido-3,5-dimethyl-adamantane impurities.

FIG. 3 illustrates a Typical Chromatogram (GC) for 1-Bromo-3,5-dimethyl-adamantane.

### DETAILED DESCRIPTION OF THE INVENTION

As used herein the term “Br-TMAD” refers to 1-bromo-3,5,7-trimethyladamantane, having the following structure:

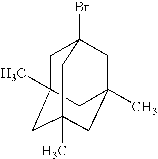

As used herein the term “Br-DMAD” refers to 1-Bromo-3,5-dimethyladamantane, having the following structure:

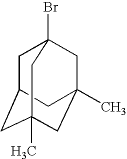

As used herein the term “Br-MMAD” refers to 1-bromo-3-methyladamantane, having the following structure:

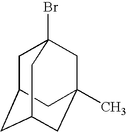

As used herein the term “Ac—NH-TMAD” refers to 1-Acetamido-3,5,7-trimethyladamantane, having the following structure:

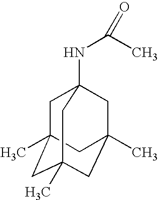

As used herein the term “Ac—NH-DMAD” refers to 1-Acetamido-3,5-dimethyladamantane, having the following structure:

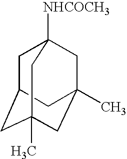

As used herein the term “Ac—NH-MMAD” refers to 1-Acetamido-3-methyladamantane, having the following structure:

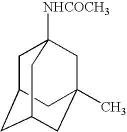

As used herein the term “Me-MMN*HCl” refers to 1-Amino-3,5,7-trimethyladamantane hydrochloride, having the following structure:

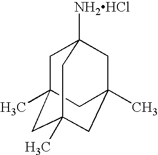

As used herein the term “MMN*HCl” refers to 1-Amino-3,5-dimethyladamantane hydrochloride, having the following structure:

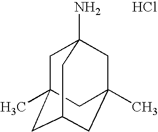

As used herein the term “DesMe-MMN*HCl” refers to 1-Amino-3-methyladamantane hydrochloride, having the following structure: 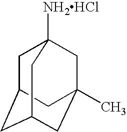
We have also discovered that during the synthesis of Memantine hydrochloride, (such as the process disclosed in co-pending U.S. application Ser. No. 11/330,681 and PCT Publication No. WO2006076562) memantine hydrochloride is produced with two impurities, namely 1-Amino-3,5,7-trimethyladamantane hydrochloride (Me-MMN*HCl) and 1-Amino-3-methyladamantane hydrochloride (DesMe-MMN*HCl). We have further discovered that these two impurities remain in the final batch even after crystallization of the hydrochloride salt from ethanol-ether.

We have discovered that the two impurities found in Memantine hydrochloride originate from the corresponding impurities in the starting material Br-DMAD as described by the following schemes:

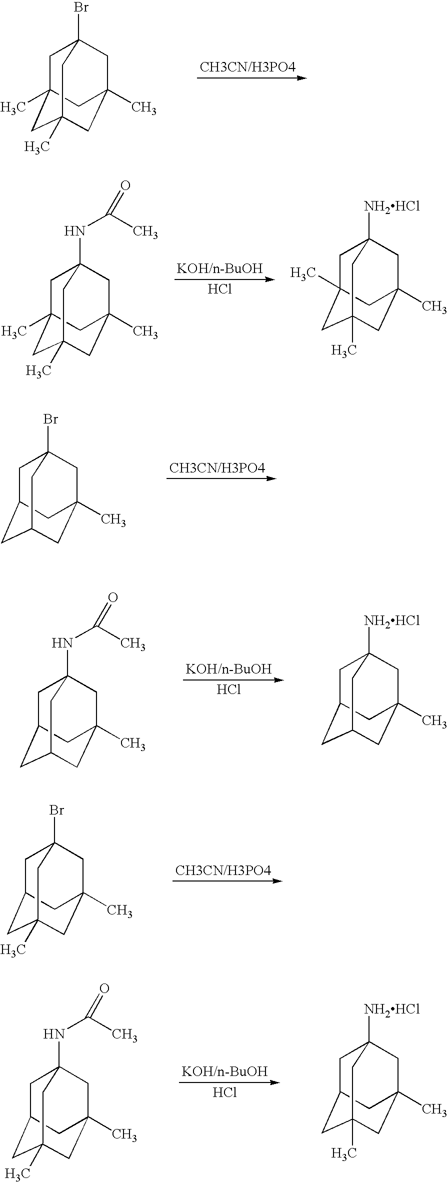

We have further found that the relative amounts of each impurity in the final product depend only on the relative amounts of Br-TMAD and Br-MMAD in Br-DMAD which is the starting material, as exemplified in the following tables:

TABLE 1

| | Impurity content (by % area GC) | |
|---|---|---|
| Compound | Br-MMAD | Br-TMAD |
| Br-DMAD raw material | 0.06% | 0.04% |
| | Ac—NH-MMAD | Ac—NH-TMAD |
| Ac—NH-DMAD raw material | 0.06% | 0.03% |
| Purification Factor from Br- | 1 | 1.33 |
| DMAD to Ac—NH-DMAD | | |
| | DesMe-MMN | Me-MMN |
| MMN*HCl | 0.04% | 0.05% |
| Purification factor from Ac—NH- | 1.5 | 0.6 |
| DMAD to MMN.HCl final solid | | |
| Purification Factor from Br- | 1.5 | 0.80 |
| DMAD to MMN*HCl final solid | | |

Tables 2 and 3 describe the impurities profiles and total purification factors (ratios of impurities in each sample) tested by Gas Chromatography (GC), when starting from Br-DMAD with different purity content:

TABLE 2

| | Impurity content (by % area GC) | |
|---|---|---|
| Compound | Br-MMAD | Br-TMAD |
| Br-DMAD raw material | 1.47% | 0.16% |
| | Ac—NH-MMAD | Ac—NH-TMAD |
| Ac—NH-DMAD final solid | 1.38% | 0.14% |
| Purification Factor from Br- | 1.065 | 1.14 |
| DMAD to Ac—NH-DMAD | | |
| | DesMe-MMN | Me-MMN |
| MMN*HCl final solid | 1.01% | 0.16% |
| Purification factor from Ac—NH- | 1.37 | 0.875 |
| DMAD to MMN.HCl final solid | | |
| Purification Factor from Br- | 1.45 | 1.00 |
| DMAD to MMN*HCl final solid | | |

TABLE 3

| | Impurity content (by % area GC) | |
|---|---|---|
| Compound | Br-MMAD | Br-TMAD |
| Br-DMAD raw material | 0.11% | 0.28% |
| | Ac—NH-MMAD | Ac—NH-TMAD |
| Ac—NH-DMAD final solid | 0.14% | 0.26% |
| Purification Factor from Br- | 0.78 | 1.07 |
| DMAD to Ac—NH-DMAD | | |
| | DesMe-MMN | Me-MMN |
| MMN*HCl final solid | 0.10% | 0.25% |
| Purification factor from Ac—NH- | 1.4 | 1.04 |
| DMAD to MMN.HCl final solid | | |
| Purification Factor from Br- | 1.1 | 1.12 |
| DMAD to MMN*HCl final solid | | |

According to the data in the above Tables, the MMN*HCl impurities content is closely connected to impurities in the starting material (Br-DMAD). This correlation can be used to quantify and reduce amount of impurities in the final product. A slightly noticeable reduction of impurity is observed in the step of converting acetyl memantine to memantine. Otherwise the relative amount of impurities remains substantially the same after each step.

TABLE 4

| | Impurity content (by % area GC) | |
|---|---|---|
| Compound | DesMe-MMN | MeMMN |
| MMN*HCl final product | <0.15% | <0.15% |
| | Ac—NH-MMAD | Ac—NH-TMAD |
| Ac—NH-DMAD | <0.15% | <0.15% |
| | Br-MMAD | Br-TMAD |
| Br-DMAD | ≦0.20% | ≦0.15% |

In one embodiment the present invention provides a process for preparing memantine HCl with less than about 0.15% area by GC of DesMe-MMN HCl and/or MeMMN HCl by measuring amount of Ac—NH-TMAD and/or Ac—NH-MMAD in batches of 1-acetamido-3,5-dimethyladamantane, selecting a batch of 1-acetamido-3,5-dimethyladamantane having less than about 0.15% area by GC of Ac—NH-TMAD and/or Ac—NH-MMAD and synthesizing memantine hydrochloride with the selected batch to obtain memantine HCl with less than about 0.15% area by GC of DesMe-MMN HCL and/or MeMMN HCl.

In another embodiment, instead of selecting a batch of 1-acetamido-3,5-dimethyladamantane, a batch of 1-bromo-3,5-dimethyladamantane is selected. Accordingly in one embodiment the present invention provides a process for reducing amount of impurities present in memantine HCl by measuring amount of Br-TMAD and/or Br-MMAD in batches of 1-bromo-3,5-dimethyladamantane, selecting batches having less than about 0.15% Br-TMAD and/or less than about 0.20% Br-MMAD area by GC and synthesizing memantine hydrochloride with the selected batch to obtain memantine HCl with less than about 0.15% of DesMe-MMN and MeMMN.

The two above embodiments can be combined. It is possible to first select a batch of 1-bromo-3,5-dimethyladamantane, synthesize 1-acetamido-3,5-dimethyladamantane, and then select a batch of 1-acetamido-3,5-dimethyladamantane as described above to synthesize memantine HCl.

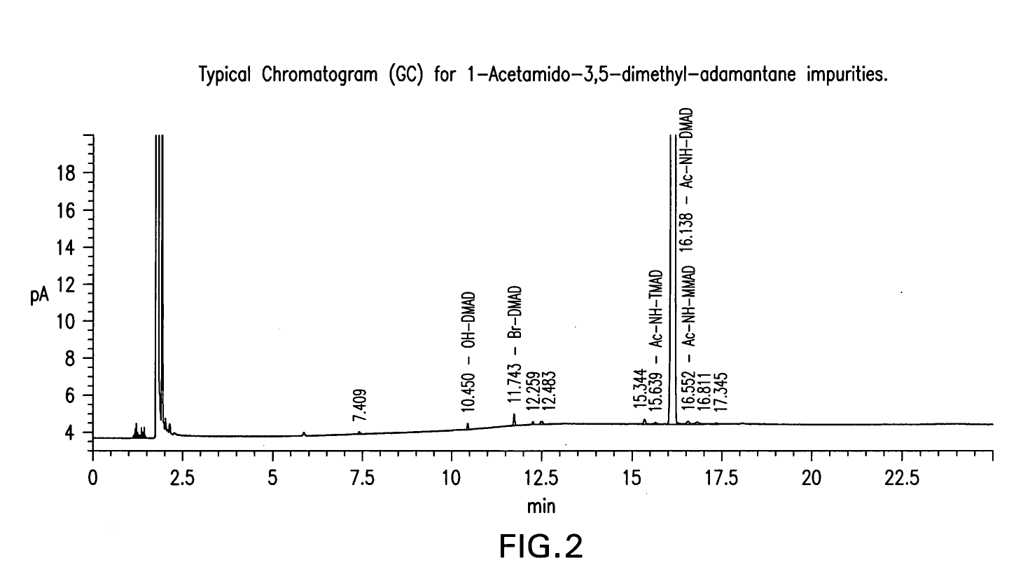

The present invention also provides isolated Ac—NH-TMAD and Ac—NH-MMAD. Preferably, these compounds are substantially free of 1-acetamido-3,5-dimethyladamantane and of each other. Preferably each of these compounds exists in a batch having less than about 1.0% by GC of 1-acetamido-3,5-dimethyladamantane. More preferably each of these isolated two compounds contains less than about 1.0% by GC of each other. These compounds can be used as a reference standard to characterize and quantify other compounds, particularly Ac—NH-DMAD. FIG. 2 illustrates the desirablity of these two compounds as a reference standard. Despite highly similar structures, the peaks for these two compounds are separate and distinct from that for Ac—NH-DMAD. The peaks for these compounds is present on each side of Ac—NH-DMAD on the chromatogram, allowing one of ordinary skill of art to identify the RRT for Ac—NH-DMAD with ease in a small area of the chromatogram. The peaks for these two compounds are much smaller than that for Ac—NH-DMAD, allowing better quantification of other impurities that exist in small amounts as well.

Accordingly in one embodiment, the present invention provides a method of identifying an impurity in a sample of Ac—NH-DMAD comprising providing a reference marker sample of Ac—NH-TMAD or Ac—NH-MMAD, or two separate reference marker samples of each; determining by chromatography the relative retention time (RRT) corresponding to Ac—NH-TMAD or Ac—NH-MMAD in a sample comprising Ac—NH-DMAD and Ac—NH-TMAD or Ac—NH-MMAD; and determining the relative retention time (RRT) of Ac—NH-TMAD or Ac—NH-MMAD in the sample by comparing the relative retention time (RRT) of the reference marker to the relative retention time (RRT) of the sample. Preferably Gas Chromatography is used.

Accordingly in another embodiment, the present invention provides a method of determining the amount of an impurity in a sample of Ac—NH-DMAD comprising measuring by chromatography the area under a peak corresponding to Ac—NH-TMAD and/or Ac—NH-MMAD in a reference standard comprising a known amount of Ac—NH-TMAD and/or Ac—NH-MMAD; measuring by chromatography the area under a peak corresponding to one or both of Ac—NH-TMAD or Ac—NH-MMAD in a sample comprising Ac—NH-DMAD and Ac—NH-TMAD and/or Ac—NH-MMAD; and determining the amount of Ac—NH-TMAD and/or Ac—NH-MMAD in the sample by comparing the area of reference standard with that of the test sample. Preferably Gas Chromatography is used.

The synthesis of 1-acetamido-3,5-dimethyladamantane and memantine HC can be carried out according to methods known in the art such as those described in WO2006/076562, incorporated herein by reference.

For example, 1-acetamido-3,5-dimethyladamantane is synthesized by combining 1-bromo-3,5-dimethyladamantane in acetonitrile with phosphoric acid. The resulting reaction mixture can be heated to complete the reaction. Heating can be carried out of about 60° C. to about reflux temperature of the solvent. Aqueous n-butanol is then added to the reaction mixture, followed by a suitable base such as Sodium/potassium hydroxide or carbonate or bicarbonate and TEA. The resulting organic phase is then separated, its pH adjusted, preferably to about 5 to about 7, and then concentrated. 1-acetamido-3,5-dimethyladamantane can then be crystallized by dissolving the concentrated organic phase in acetone, methanol, ethanol and IPA and adding water to precipitate the crystals.

The above synthetic method can be carried out to synthesize and crystallize Ac—NH-TMAD and Ac—NH-MMAD by starting with Br-MMAD or Br-TMAD.

To prepare memantine HCl, the 1-acetamido-3,5-dimethyladamantane can then be combined with n-butanol, pentanol, ethylene glycol and a base such as potassium or sodium hydroxide and heated to accelerate the hydrolysis of the acetyl group. Heating can be carried out to about 128-132° C. The resulting solution can then be cooled, such as to about 45-50° C. and water added thereto to form a biphasic system. The organic phase is then separated and its pH can be adjusted, such as to about 10.5-11 by addition of an acid, preferably HCl. The organic phase can be washed with water. HCl is then added to the organic phase to obtain a solution. To crystallize the memantine HCl, the organic phase can be concentrated to obtain a residue, the residue added to ethyl acetate, acetone and buthylacetate, and then cooled, preferably to a temperature of about 0±5° C. to obtain memantine HCl. The memantine HCl can be dried such as by heating to a temperature of about 55±5° C.

The above synthetic method can be carried out to synthesize and crystallize DesMe-MMN HCl and MeMMN HCl by starting with Ac—NH-TMAD and Ac—NH-MMAD.

Preferably, the GC methodology used with any of the above embodiments with regard to Ac—NH-DMAD, Ac—NH-TMAD and Ac—NH-MMAD includes combining Ac—NH-DMAD sample with methanol, to obtain a solution; injecting the solution into a 30 m×0.32 mm×0.50 cm RTX-35 (or similar) column; eluting the sample from the column at about 25 min using nitrogen as a carrier gas, and measuring the Ac—NH-TMAD or Ac—NH-MMAD content in the relevant sample with a Flame Ionization Detector (FID).

Preferably, the GC methodology used with any of the above embodiments with regard to Br-TMAD, Br-MMAD and Br-DMAD includes the steps combining Br-DMAD sample with methylene chloride, to obtain a solution; injecting the solution into a gas chromatograph with a 30 m×0.32 mm×0.50 μm RTX-35 (or similar) column; eluting the sample from the column at about 25 min using nitrogen as a carrier gas, and measuring the Br-TMAD or Br-MMAD content in the relevant sample with a Flame Ionization Detector (FID).

Pharmaceutical compositions of the present invention contain memantine HCl with at least one pharmaceutically acceptable excipient. These pharmaceutical compositions are prepared by combining memantine HCl prepared by the processes of the present invention with an excipient. The memantine HCl contains less than about 0.15% DesMe-MMN HCl and/or MeMMN HCl as measured by GC.

Accordingly, in one embodiment the present invention provides a process for preparing a pharmaceutical composition comprising memantine hydrochloride having less than about 0.15% area by GC of Me-MMN*HCl and/or DesMe-MMN*HCl, comprising obtaining one or more batches of the compound memantine hydrochloride, measuring the level of Me-MMN*HCl and/or DesMe-MMN*HCl in each of the batches, selecting a batch of memantine hydrochloride having a level of either Me-MMN*HCl or DesMe-MMN*HCl of less than about 0.15% area by GC; and preparing with the selected batches a pharmaceutical composition comprising memantine hydrochloride and at least one pharmaceutically acceptable excipient.

One excipient can be a diluent. Diluents increase the bulk of a solid pharmaceutical composition and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form like a tablet may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flow properties of non-compacted solid composition and improve the accuracy of dosing. Excipients that may function as glidants include colloidal silicon dioxide, magnesium trisilicate, powdered cellulose, starch, talc and tribasic calcium phosphate.

When a dosage form such as a tablet is made by compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease release of the product from the die. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, at the above Tacrolimus and at least one excipient are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

A liquid composition according to the present invention may also contain a buffer such as guconic acid, lactic acid, citric acid or acetic acid, sodium guconate, sodium lactate, sodium citrate or sodium acetate.

Selection of excipients and the amounts to use may be readily determined by the formulation scientist based upon experience and consideration of standard procedures and reference works in the field.

The solid compositions of the present invention include powders, granulates, aggregates and compacted compositions. The dosage forms include dosage forms suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well known in the pharmaceutical arts.

Dosage forms include solid dosage forms like tablets, powders, capsules, suppositories, sachets, troches and lozenges as well as liquid syrups, suspensions and elixirs.

A dosage form of the present invention can be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant. An especially preferred capsule filling contains, in addition to one or more of the fluvastatin sodium crystalline forms of this invention, the excipients magnesium stearate, microcrystalline cellulose, pregelatinized starch, sodium lauryl sulfate and talc.

Another dosage form of this invention is a compressed tablet that contains, in addition to one or more of the fluvastatin sodium crystalline forms of this invention, the excipients microcrystalline cellulose, hydroxypropyl cellulose, hydroxypropylmethyl cellulose, potassium bicarbonate, povidone, magnesium stearate, iron oxide yellow, titanium dioxide, and polyethylene glycol 8000.

The active ingredient and excipients may be formulated into compositions and dosage forms according to methods known in the art.

A composition for tableting or capsule filing may be prepared by wet granulation. In wet granulation some or all of the active ingredients and excipients in powder form are blended and then further mixed in the presence of a liquid, typically water, that causes the powders to clump up into granules. The granulate is screened and/or milled, dried and then screened and/or milled to the desired particle size. The granulate may then be tableted or other excipients may be added prior to tableting such as a glidant and or lubricant.

A tableting composition may be prepared conventionally by dry blending. For instance, the blended composition of the actives and excipients may be compacted into a slug or a sheet and then comminuted into compacted granules. The compacted granules may be compressed subsequently into a tablet.

As an alternative to dry granulation, a blended composition may be compressed directly into a compacted dosage form using direct compression techniques. Direct compression produces a more uniform tablet without granules. Excipients that are particularly well suited to direct compression tableting include microcrystalline cellulose, spray dried lactose, dicalcium phosphate dihydrate and colloidal silica. The proper use of these and other excipients in direct compression tableting is known to those in the art with experience and skill in the particular formulation challenges of direct compression tableting.

A capsule filling of the present invention may comprise any of the aforementioned blends and granulates that were described with reference to tableting, only they are not subjected to a final tableting step.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Example 1

### GC Method for Memantine Hydrochloride Impurities

GC was performed with a PTA-5 Base Deactivated poly (5% diphenyl/95% dimethylsiloxane) column 30 m in length, 0.32 m in diameter, and having 0.50 μm film thickness and a flame ionization detector. The injector temperature was 280° C. and the detector temperature was 300° C. The carrier gas was either nitrogen or helium and the flow rate was 2.0 mL per minute. The split ratio was 20/1. The oven temperature was set at 100° C. for the first 11.67 minutes and at 200° C. between 11.67 minutes and 25 minutes. The diluent for the samples was pyridine (Aldrich cat num P57506) and the volume of each sample injected into the GC was 1 μL. The syringe used to inject the samples into the GC was washed with a 1:1 mixture of 0.1N NaOH and acetonitrile between each sample.

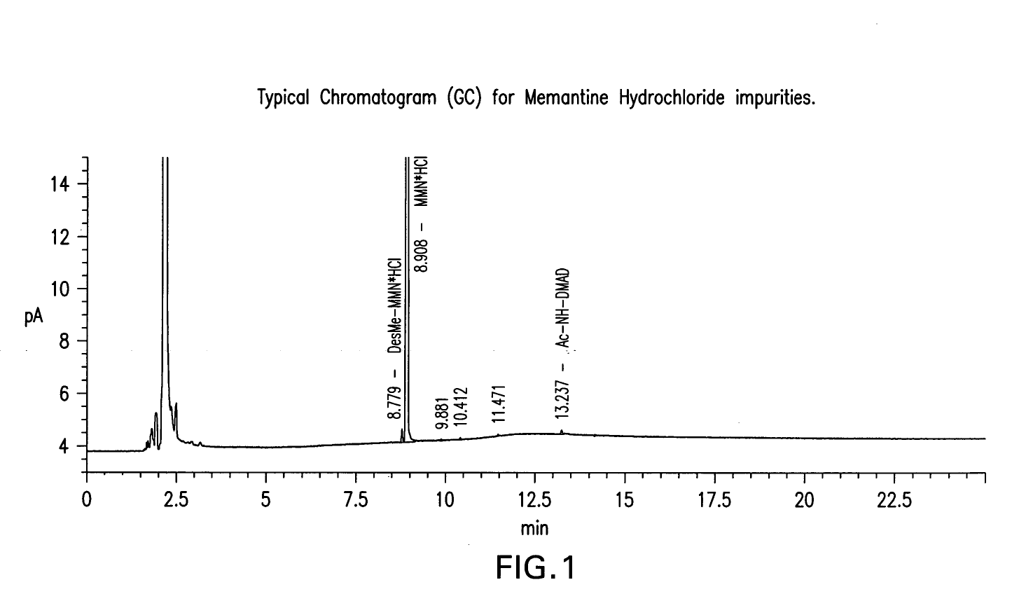

Temperature and flow rate may be varied in order to achieve the required system suitability. The DL was 0.03% and the QL was 0.05%. Typical results were illustrated in FIG. 1.

### Example 2

### GC Method for 1-Acetamido-3,5-dimethyl-adamantane Impurities

GC was performed with a RTX-35 35% dIPh-polysiloxane RESTEK cat. Num. 10439 or equivalent column 30 m in length, 0.32 m in diameter, and having 0.50 μm film thickness and a flame ionization detector. The injector temperature was 280° C. and the detector temperature was 300° C. The carrier gas was either nitrogen and the flow rate was 2.0 mL per minute. The split ratio was 20/1. The oven temperature was set at 100° C. for the first 11.67 minutes and at 200° C. between 11.67 minutes and 25 minutes.

Temperature and flow rate may be varied in order to achieve the required system suitability. The DL is 0.03% and the QL is 0.05%. Typical results were illustrated in FIG. 2.

### Example 3

### GC Method for 1-Bromo-3,5-dimethyl-adamantane (Br-DMAD)

GC was performed with a RTX-35 35% dIPh-polysiloxane RESTEK cat. Num. 10439 or equivalent column 30 m in length, 0.32 m in diameter, and having 0.50 μm film thickness and a flame ionization detector. The injector temperature was 280° C. and the detector temperature was 300° C. The carrier gas was either nitrogen and the flow rate was 2.0 mL per minute. The split ratio was 20/1. The oven temperature was set at 100° C. for the first 11.67 minutes and at 200° C. between 11.67 minutes and 25 minutes.

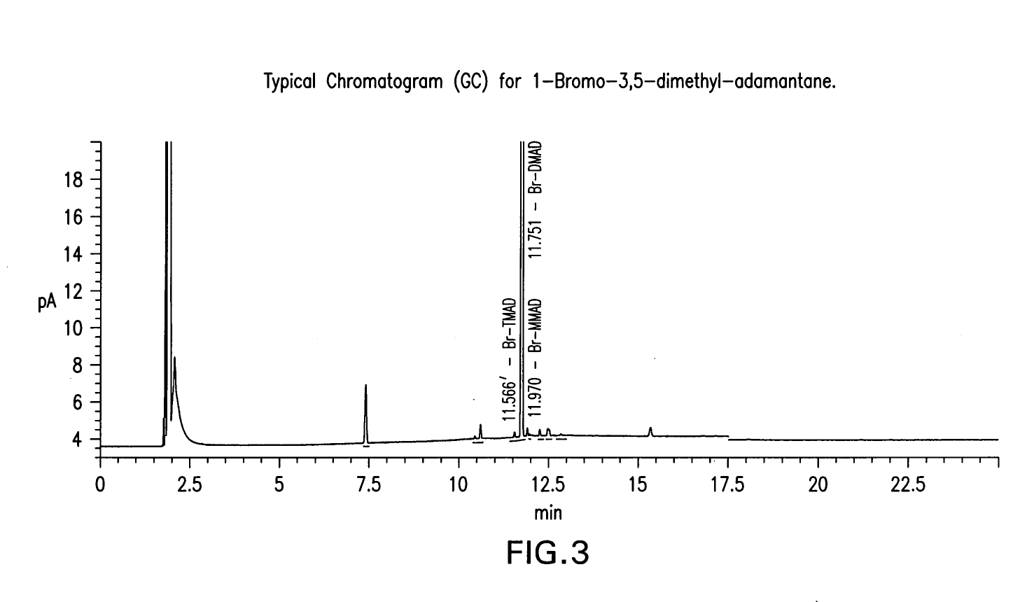

Temperature and flow rate may be varied in order to achieve the required system suitability. Typical results are illustrated in FIG. 3.

### Example 4

### 1-Acetamido-3,5-dimethyladamantane (Ac—NH-DMAD) Synthesis

500 g of 1-bromo-3,5-dimethyladamantane and 393 g (500 ml) of acetonitrile were loaded into a 5-liter reactor equipped with condenser, mechanical stirrer, thermometer at 20-25° C. under nitrogen. After about 15-20 minutes, 806 g of 75% phosphoric acid was added. After addition of the phosphoric acid, the internal temperature of the reactor rose to 30-32° C. and a biphasic system was obtained. The biphasic system was warmed to 87±2° C. over about 30 min (slight reflux) and the temperature maintained for 3-3.5 hrs. A monophasic system was then obtained. At this point, the reaction was complete (sum of 1-hydroxy-3,5-dimethyladamantane and 1-bromo-3,5-dimethyladamantane less than 1%).

n-butanol (1000 ml) and water (770 ml) were added to the monophasic system and the system was cooled to 20-25° C. Sodium hydroxide 30% (337.5 g) was added and the temperature rises to 40-45° C. Two phases form and the phases were separated at 35-40° C. The aqueous phase was discarded and water (385 g) was added to the organic phase to form a biphasic system. Then NaOH 30% (337.5 g) was added, while maintaining the temperature at 40-45° C. and adjusting the pH to 5.5-7. The phases were separated at 40-45°. The organic phase was concentrated under vacuum (res. pressure 45-50 mmHg, external temperature 80-85° C., internal temperature 40-70° C.) until a residual volume of 600-650 ml was obtained. After cooling to 55-60° C., acetone (474 g) was added. The resulting suspension was warmed to reflux (62-63° C.) until complete dissolution was obtained. After cooling to 50° C., water (2000 ml) was slowly added (over about 30 min) at 45-50° C. and crystallization of 1-acetamido-3,5-dimethyladamantane occurred. At the beginning some oil separated. Usually spontaneous crystallization occurs, but if not, seeding with about 0.2-0.3% of solid is necessary. After 1.5-2 hrs at 18±3° C. solid was filtered, washed with water and dried at 45-50° C. for 15 hrs.

Dry weight: 435 g, Yield: 95.5%, Purity: 99.84% by GC.

### Example 5

### Memantine Hydrochloride Synthesis

486 g (600 ml) of n-butanol, 150 g of 1-acetamido-3,5-dimethyladamantane and 241 g of 89.9% potassium hydroxide are added to a 2-liter reactor equipped with a condenser, a mechanical stirrer, and a thermometer at 20-25° C. under nitrogen. After addition, the internal temperature rises to 40-45° C. without external cooling. The resulting suspension is heated to 128-132° C. over 20-30 min and a solution is obtained. After 10 hrs at 128-132° C. (no reflux), the reaction is complete (unreacted 1-acetamido-3,5-dimethyladamantane less than 1%).

After cooling to 45-50° C., water (450 ml) is added to form a biphasic system. After stirring (5 min) and standing (15 min) at 20-25° C., the phases are separated. The aqueous phase is discarded and water (225 ml) is added to organic phase to form a biphasic system and pH is brought to 10.5-11 with 37% hydrochloric acid (10 g). After stirring (5 min) and standing (15 min) at 20-25° C., the phases are separated. Water (225 ml) is added to the organic phase to form a biphasic system and after stirring (5 min) and standing (15 min) at 20-25° C., the phases are separated. To the organic phase, 37% hydrochloric acid (66.9 g) is added and the solution is filtered on paper filter. The obtained solution is concentrated under vacuum until a residual volume of 360 ml is obtained (a semisolid but well stirrable mixture) and internal temperature is 50-55° C. At this point, after cooling to 45-50° C., ethyl acetate (750 ml) is added. The obtained suspension is cooled to 03° C. and after 3 hrs it is filtered and solid washed three times with ethyl acetate (90 ml each). Wet white solid is dried under vacuum at 55-60° C. for 15 hrs. Dry weight: 138.7 g, Yield: 95%, Purity: 99.97% by GC.

### Example 6

### 1-Acetamido-3,5,7-dimethyladamantane (Ac—NH-TMAD) Synthesis

4 g of 1-bromo-3,5,7-trimethyladamantane, 12 g (15 ml) of acetonitrile, and 7 g of 75% phosphoric acid are added to a 50 ml reactor equipped with condenser, mechanical stirrer, thermometer at 20-25° C. and under nitrogen. After addition, the internal temperature rises to 30-32° C. The obtained biphasic system is warmed to 87±2° C. over about 30 min (slight reflux) and the temperature maintained for 17 hrs. During the course of the reaction, a monophasic system is obtained. At this point, the reaction is complete.

n-butanol (15 ml), toluene (15 ml) and water (15 ml) are added and the resulting biphasic system is cooled to 20-25° C. Sodium hydroxide 30% is added to reach pH 6-7 and the temperature rises to 40-45° C. Phases are separated at 35-40° C. and the aqueous 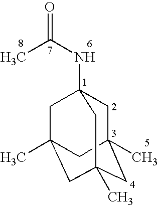
phase is discarded. Water (15 ml) is loaded to the organic phase and, after stirring and standing, the phases are separated at 40-45°. The organic phase is concentrated under vacuum (res. pressure 45-50 mmHg, external temperature 80-85° C., internal temperature 40-70° C.) until a residual volume of 6-6.5 ml is obtained. After cooling to 55-60° C., acetone (30 ml) is added. The resulting suspension is warmed to reflux (62-63° C.) until complete dissolution is obtained. After cooling to 50° C., water (50 ml) is slowly added and crystallization of 1-acetamido-3,5,7-dimethyladamantane occurs. After 1.5-2 hrs at 18±3° C., the solid is filtered, washed with water and dried at 45-50° C. for 15 hrs. Dry weight: 3.3 g

¹H NMR is reported:

¹H-NMR in CDCl₃ (298K)

| No. of carbon | Chem. Shift of hydrogens | Multiplicity | |
|---|---|---|---|
| atom | attached to carbon atom, ppm | (Integration) | J, Hz |
| 6 | 5.31 | bs(1H) | — |
| 8 | 1.87 | s(3H) | — |
| 2 | 1.54 | s(6H) | — |
| 4 | 1.10 | d(3H) | 11.9 |
| 4 | 1.00 | d(3H) | 11.9 |
| 5 | 0.83 | s(9H) | — |

### Example 7

### 1-Amino-3,5,7-trimethyladamantane Hydrochloride (Me-MMN*HCl) Synthesis

16.2 g (20 ml) of n-butanol, 2.3 g of 1-acetamido-3,5,7-dimethyladamantane, and 3.6 g of 89.9% potassium hydroxide were added to a 50 ml reactor equipped with a condenser, a mechanical stirrer, and a thermometer at 20-25° C. under nitrogen. After addition, the internal temperature rises to 40-45° C. without external cooling. The resulting suspension is heated to 128-132° C. over 20-30 min and a solution is obtained. After 15 hrs at 128-132° C. (slight reflux), the reaction is considered to be complete (unreacted 1-acetamido-3,5,7-dimethyladamantane less than 5%).

After cooling to 45-50° C., water (20 ml) is added to form a biphasic system. After stirring (5 min) and standing (15 min) at 20-25° C. phases are separated. The aqueous phase is discarded and the organic phase is washed with water (2×20 ml). The obtained organic solution is acidified with HCl to pH 1 and the solution is concentrated under vacuum until a semisolid is obtained. At this point, after cooling to 45-50° C., ethyl acetate (40 ml) is added. The obtained suspension is cooled to 0±3° C. and after 3 hrs the suspension is filtered and the recovered solid is washed three times with ethyl acetate (6 ml each). Wet white solid is dried under vacuum at 55-60° C. for 15 hrs. Dry weight, 1.93 g.

¹H NMR is reported:

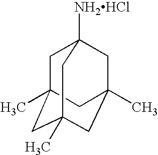

¹H-NMR in CD₃OD (298K)

| No. of carbon | Chem. Shift of hydrogens | Multiplicity | |
|---|---|---|---|
| atom | attached to carbon atom, ppm | (Integration) | J, Hz |
| SRP | 4.84 | bs(nH) | — |
| SRP | 3.30 | bs(nH) | — |
| 2 | 1.45 | s(6H) | — |
| ½ 4 | 1.16 | s(6H) | — |
| 5 | 0.93 | s(9H) | — |

### Example 8

### 1-Acetamido-3-methyladamantane (Ac—NH-MMAD) Synthesis

5.5 g of 1-bromo-3-methyladamantane, 16 g (20 ml) of acetonitrile, and 75% phosphoric acid were added to a 50 ml reactor equipped with condenser, mechanical stirrer, and thermometer at 20-25° C. under nitrogen. After addition, the internal temperature rises to 30-32° C. The obtained biphasic system is warmed to 87±2° C. over about 30 min (slight reflux) and the temperature maintained for 18 hrs. During the course of the reaction, a monophasic system is obtained. At this point, the reaction is complete.

n-butanol (15 ml), toluene (15 ml) and water (15 ml) are added to form a biphasic system and the system is cooled to 20-25° C. Sodium hydroxide 30% is added to reach pH 6-7 and the temperature rises to 40-45° C. The phases are separated at 35-40° C. The aqueous phase is discarded. Water (15 ml) is added to the organic phase to form a biphasic system and, after stirring and standing, the phases are separated at 40-45°. The organic phase is concentrated under vacuum (res. pressure 45-50 mmHg, external temperature 80-85° C., internal temperature 40-70° C.) until a residual volume of 6-6.5 ml is obtained. After cooling to 55-60° C., acetone (5 ml) is added and water (50 ml) is slowly added. Crystallization of 1-acetamido-3-methyladamantane occurs. After 1.5-2 hrs at 18±3° C., the solid is filtered, washed with water and dried at 45-50° C. for 15 hrs. Dry weight. 3.96 g.

¹H NMR is reported:

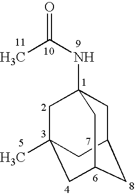

¹H-NMR in CDCl₃ (298K)

| No. of carbon | Chem. Shift of hydrogens | Multiplicity | |
|---|---|---|---|
| atom | attached to carbon atom, ppm | (Integration) | J, Hz |
| 9 | 5.56 | bs(1H) | — |
| 6 | 2.03 | m(2H) | — |
| 7* | 1.89 | m(2H) | — |
| 11 | 1.84 | s(3H) | — |
| 7* | 1.83 | m(2H) | — |
| 2 | 1.65 | s(2H) | — |
| 8 | 1.54 | m(1H) | (12.4) |
| 8 | 1.47 | m(1H) | (12.4) |
| 4* | 1.34 | m(4H) | — |
| Imp | 1.19 | s(nH) | — |
| Imp | 0.79 | s(nH) | — |
| 5 | 0.76 | s(3H) | — |

### Example 9

### 1-Amino-3-methyladamantane hydrochloride (DesMe-MMN.HCl) Synthesis

16.2 g (20 ml) of n-butanol, 2.8 g of 1-acetamido-3-methyladamantane and 7 g of 89.9% potassium hydroxide are added to a 50 ml reactor equipped with a condenser, a mechanical stirrer, and a thermometer at 20-25° C. and under nitrogen. After addition, the internal temperature rises to 40-45° C. without external cooling. The resulting suspension is heated to 128-132° C. over 20-30 min and a solution is obtained. After 12 hrs at 128-132° C. (slight reflux), the reaction is complete.

After cooling to 45-50° C., water (20 ml) is added to form a biphasic system. After stirring (5 min) and standing (15 min) at 20-25° C., the phases are separated. The organic phase is washed with water (4×20 ml). The obtained organic solution is then acidified to pH 1 and concentrated under vacuum until a semisolid is obtained. At this point, after cooling to 45-50° C., ethyl acetate (40 ml) is added. The obtained suspension is cooled to 0±3° C. and after 3 hrs the suspension is filtered and the recovered solid washed three times with ethyl acetate (6 ml each). Wet white solid is dried under vacuum at 55-60° C. for 15 hrs. Dry weight, 2.2 g.

¹H NMR is reported:

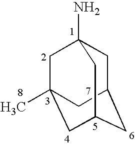

¹H-NMR in CDCl₃ (298K)

| No. of carbon | Chem. Shift of hydrogens | Multiplicity | |
|---|---|---|---|
| atom | attached to carbon atom, ppm | (Integration) | J, Hz |
| SRP | 4.85 | bs(nH) | — |
| SRP | 3.30 | bs(nH) | — |
| 5 | 2.20 | m(2H) | (2.8) |
| ½ 7 | 1.84 | bd(2H) | 11.8 |
| ½ 7 | 1.78 | bd(2H) | 12.9 |
| 6 | 1.65 | m(2H) | — |
| 2 | 1.59 | bs(2H) | — |
| ½ 4 | 1.50 | bd(2H) | 12.9 |
| ½ 4 | 1.46 | bd(2H) | 14.0 |
| 8 | 0.90 | s(3H) | — |

### Example 10

### Crystallization from Diethylether/Ethanol

10 g of Memantine hydrochloride was suspended in 100 ml of diethylether and the suspension was warmed to reflux. Ethanol was added slowly till a solution was obtained (150 mL). The solution was cooled to 0° C. and crystallization occurred. The suspension was stirred at 0° C. for two hours and then the solid was filtered and washed with diethylether/ethanol mixture 2:3. Obtained solid was dried at atmospheric conditions to obtain 5 g.

What is claimed is:

1. A process for preparing memantine HCl having less than about 0.15% of one or both of Ac—NH-TMAD and Ac—NH-MMAD comprising measuring an amount of at least one or both of N-acetyl-1-amino-3,5,7-trimethyladamantane (Ac—NH-TMAD) and N-acetyl-1-amino-3-methyladamantane (Ac—NH-MMAD) in a batch of 1-acetamido-3,5-dimethyladamantane, selecting a batch of 1-acetamido-3,5-dimethyladamantane having less than about 0.15% of one or both of Ac—NH-TMAD or Ac—NH-MMAD and converting the selected batch of 1-acetamido-3,5-dimethyladamantane to memantine HCl containing less than about 0.15% of at least one of DesMe-MMN HCl or MeMMN HCl.

2. A process according to claim 1 wherein the conversion comprises hydrolysis of the acetyl group of 1-acetamido-3,5-dimethyladamantane and subsequent reaction with hydrochloric acid.

3. A process according to claim 2 wherein the hydrolysis comprises reaction of the 1-acetamido-3,5-dimethyladamantane with a base in the presence of a solvent.

4. A process for preparing memantine HCl containing less than about 0.15% of at least one of DesMe-MMN HCl or MeMMN HCl comprising measuring an amount of one or both of 1-bromo-3,5,7-trimethyladamantane (Br-TMAD) or 1-bromo-3-methyladamantane (Br-MMAD) in a batch of 1-bromo-3,5-dimethyladamantane, selecting a batch having one or both of less than about 0.15% of Br-TMAD or less than about 0.20% area Br-MMAD and converting the batch of 1-bromo-3,5-dimethyladamantane to memantine HCl containing less than about 0.15% of at least one of DesMe-MMN HCl and MeMMN HCl.

5. A process according to claim 4 wherein the conversion comprises:
(a) reacting 1-bromo-3,5-dimethyladamantane with acetonitrile and phosphoric acid to produce 1-acetamido-3,5-dimethyladamantane,
(b) hydrolysis of the acetyl group of 1-acetamido-3,5-dimethyladamantane to produce memantine; and
(c) reacting the memantine with HCl.

6. A process according to claim 5 wherein the hydrolysis comprises reaction of the 1-acetamido-3,5-dimethyladamantane with a base in the presence of a solvent.

7. A process for reducing amount of impurities present in memantine HCl comprising measuring an amount of at least one or both of 1-bromo-3,5,7-trimethyladamantane (Br-TMAD) and 1-bromo-3-methyladamantane (Br-MMAD) in a batch of 1-bromo-3,5-dimethyladamantane, selecting a batch having at least one of less than about 0.15% Br-TMAD or less than about 0.20% area Br-MMAD as measured by gas chromatography, and converting the batch of 1-bromo-3,5-dimethyladamantane to 1-acetamido-3,5-dimethyladamantane; measuring an amount of at least one of N-acetyl-1-amino-3,5,7-trimethyladamantane (Ac—NH-TMAD) and N-acetyl-1-amino-3-methyladamantane (Ac—NH-MMAD) in a batch of 1-acetamido-3,5-dimethyladamantane, selecting a batch of 1-acetamido-3,5-dimethyladamantane having less than about 0.15% area by gas chromatography of at least one of Ac—NH-TMAD and Ac—NH-MMAD and converting the selected batch of 1-acetamido-3,5-dimethyladamantane to memantine HCl containing less than about 0.15% of at least one of DesMe-MMN HCl and MeMMN HCl.

8. A process according to claim 7 wherein the conversion of 1-bromo-3,5-dimethyladamantane to 1-acetamido-3,5-dimethyladamantane comprises reaction with acetonitrile and phosphoric acid.

9. A process according to claim 7 wherein the conversion of 1-acetamido-3,5-dimethyladamantane to memantine HCl comprises hydrolysis of the acetyl group and subsequent reaction with hydrochloric acid.

10. A process according to claim 9 wherein the hydrolysis comprises reaction of the 1-acetamido-3,5-dimethyladamantane with a base in the presence of a solvent.

11. Isolated N-acetyl-1-amino-3,5,7-trimethyladamantane (Ac—NH-TMAD).

12. The isolated Ac—NH-TMAD of claim 11, wherein the Ac—NH-TMAD has at most 1% of 1-acetamido-3,5-dimethyladamantane

13. Isolated N-acetyl-1-amino-3-methyladamantane (Ac—NH-MMAD).

14. The isolated Ac—NH-MMAD of claim 13, wherein the Ac—NH-TMAD has at most 1% of 1-acetamido-3,5-dimethyladamantane.

15. A method of determining the amount of an impurity in a sample of N-acetyl-1-amino-3,5-dimethyladamantane (Ac—NH-DMAD) comprising measuring by chromatography the area under a peak corresponding to at least one of N-acetyl-1-amino-3,5,7-trimethyladamantane (Ac—NH-TMAD) and N-acetyl-1-amino-3-methyladamantane (Ac—NH-MMAD) in a reference standard comprising a known amount of one or both of Ac—NH-TMAD and Ac—NH-MMAD; measuring by chromatography the area under a peak corresponding to Ac—NH-TMAD or Ac—NH-MMAD in a sample comprising Ac—NH-DMAD and at least one of Ac—NH-TMAD or Ac—NH-MMAD; and determining the amount of at least one of Ac—NH-TMAD and Ac—NH-MMAD in the sample by comparing the area of reference standard with that of the test sample.

17. The process of claim 16, wherein the chromatography is gas chromatography.

18. A method of identifying an impurity in a sample of Ac—NH-DMAD comprising providing a reference marker sample of Ac—NH-TMAD or Ac—NH-MMAD, or two separate samples of each; determining by chromatography the relative retention time (RRT) corresponding to at least one of Ac—NH-TMAD or Ac—NH-MMAD in a sample comprising Ac—NH-DMAD, and Ac—NH-TMAD or Ac—NH-MMAD; and determining the relative retention time (RRT) of Ac—NH-TMAD or Ac—NH-MMAD in the sample by comparing the relative retention time (RRT) of the reference marker to the relative retention time (RRT) of the sample.

19. The process of claim 18, wherein the chromatography is gas chromatography.

20. A process for preparing a pharmaceutical composition comprising memantine hydrochloride having less than about 0.15% of one or both Me-MMN*HCl or DesMe-MMN*HCl, comprising obtaining one or more batches of the compound memantine hydrochloride, measuring the level of either Me-MMN*HCl or DesMe-MMN*HCl in each of the samples, selecting a batch of memantine hydrochloride having less than about 0.15% of one or both of Me-MMN*HCl or DesMe-MMN*HCl, based on the measurement of the samples from the batches; and preparing from the selected batch a pharmaceutical composition comprising memantine hydrochloride and at least one pharmaceutically acceptable excipient.
